(19) United States
(12) Patent Application Publication
(10) Pub. No.: US 2016/0083637 A1
(43) Pub. Date: Mar. 24, 2016

(54) METHOD FOR FREEZING AND THAWING ICE PARTICULATE FLUIDS

(71) Applicant: THE TRUSTEES OF THE UNIVERSITY OF PENNSYLVANIA, Philadelphia, PA (US)

(72) Inventor: JOSHUA LAMPE, JENKINTOWN, PA (US)

(21) Appl. No.: 14/889,678

(22) PCT Filed: May 6, 2014
(86) PCT No.: PCT/US14/36917

§ 371 Date: Nov. 6, 2015

Related U.S. Application Data
(60) Provisional application No. 61/819,829, filed on May 6, 2013.

(51) Int. Cl. C09K 5/06; A61F 7/02; H05B 6/80; A61K 33/14; A61K 33/00

(52) U.S. Cl. CPC C09K 5/066; A61K 33/14; A61K 33/00; H05B 6/806; A61F 7/02

(57) ABSTRACT

A frozen particulate slurry comprises a first component and a second component which are capable of forming a eutectic mixture. The slurry is formed by cooling a composition comprising the first and second components to a temperature below the freezing point of the first component to form particles of the first component, and then cooling to freeze a eutectic mixture of the first component and the second component. A method of thawing the frozen particulate slurry comprises heating the slurry, and an apparatus for thawing the frozen particulate slurry comprises a container for holding the particulate slurry, a heat source, and an agitator. A method of inducing therapeutic hypothermia in a patient comprises heating the frozen particulate slurry and administering the particulate slurry to the patient in an amount sufficient to locally or systemically induce therapeutic hypothermia.

This application is related to and claims the benefit of priority of U.S. Provisional Application No. 61/819,829, entitled METHOD FOR FREEZING AND THAWING ICE PARTICULATE FLUIDS, filed on 6 May 2013, the contents of which are incorporated herein by reference.

### FIELD OF THE INVENTION

The present invention relates generally to the field of coolants. More specifically, the invention relates to particulate coolants, such as ice particulate coolants, and methods of freezing and thawing particulate coolants and treatment methods using the coolants.

### BACKGROUND OF THE INVENTION

In the art of coolants, two-phase coolants have been developed to take advantage of the thermodynamic benefits of the phase change. For example, an ice particulate two-phase coolant comprises ice in a fluid. The ice particulate offers greater cooling capacity than the fluid alone because the ice undergoes a phase transition from a solid to a liquid.

The formation of two-phase coolants has been disclosed, for example, in U.S. Pat. No. 8,117,854, which is incorporated by reference herein, in its entirety and for all purposes.

There is great interest in two-phase coolants for use in the food and medical industries. For example, ice particulate slurries can be used for inducing hypothermia in a patient, either locally or systemically, for medical procedures such as surgical procedures or other procedures such as cardiopulmonary resuscitation (CPR). Two-component coolants have also found use for cooling perishable items, such as food or beverages, chemicals, drugs and pharmaceutical compounds, cells, tissues, biological fluids, organs, and the like. Two-component coolants have also found use for building refrigeration and fire suppression.

One obstacle to the use of two-phase coolants affecting the availability, production, sale, and use of two-coolants is the challenge associated with maintaining the fluidity of two-phase coolants.

When left undisturbed, two-phase coolants tend to separate due to the different densities of the liquid and solid phases. The solid phase within two-phase coolants also agglomerate due to the constant phase change at the solid-liquid boundary.

Equipment to prepare and maintain the consistency and fluidity of two-phase coolants exists, but such equipment is typically large and requires a significant amount of power to operate. The size and power requirements of the equipment prevents two-phase coolants from more widespread use. Therefore, two-phase coolants are often limited to larger installations with the space and power supply to support the equipment.

### SUMMARY OF THE INVENTION

It is desirable to provide two-phase coolants at the location where such coolants are needed. For example, emergency medical technicians (EMTs) responding to calls requiring the administration of CPR must provide all of their equipment in an ambulance. Thus, a need exists for equipment and methods for producing two-phase coolants that are portable and can quickly produce two-phase coolants.

In one aspect, the present invention relates to a composition comprising a plurality of particles suspended in a eutectic solid, wherein the plurality of particles consisting essentially of a first component, the eutectic solid comprises a eutectic mixture of the first component and a second component, and the composition is at a temperature below the eutectic temperature of the eutectic mixture.

Another aspect of the present invention relates to a method of freezing a composition comprising:
cooling a composition comprising a first component and a second component, wherein the first component and the second component are capable of forming a eutectic mixture and the first component has a freezing point higher than the eutectic temperature of the eutectic mixture, to a temperature below the freezing point of the first component and above the eutectic temperature for a time sufficient to freeze at least a portion of the first component to form a particulate slurry consisting essentially of particles of the first component; and
cooling the particulate slurry to a temperature below the eutectic temperature to freeze the remainder of the composition.

An aspect of the present invention also relates to a method of thawing a frozen particulate slurry comprising:
heating a frozen particulate slurry comprising particles of a first component surrounded by a eutectic solid comprising the first component and a second component, wherein the heating melts at least a portion of the eutectic solid and forms a particulate slurry comprising the particles of the first component in a solution of the first component and the second component.

Yet another aspect of the present invention relates to an apparatus for controlled thawing of a particulate slurry comprising:
a container for holding the particulate slurry;
a heat source; and
an agitator for mixing the particulate slurry;

wherein the heat source is capable of evenly providing heat throughout the particulate slurry.

Another aspect of the present invention relates to a method of inducing therapeutic hypothermia in a patient, comprising:
heating a frozen particulate slurry comprising particles of a first component surrounded by a eutectic solid comprising the first component and a second component, wherein the heating melts at least a portion of the eutectic solid and forms a particulate slurry comprising the particles of the first component in a solution of the first component and the second component; and
administering the particulate slurry to the patient in an amount sufficient to locally or systemically induce therapeutic hypothermia.

### BRIEF DESCRIPTION OF THE DRAWINGS

FIG. 1 is a schematic representation of an apparatus for controlled thawing of a particulate slurry according to an embodiment of the invention.

### DETAILED DESCRIPTION OF THE INVENTION

As used herein, the term “two-phase coolant” refers to a composition comprising two phases of matter (e.g., solid and liquid) that may be used to absorb heat. Unless otherwise specified, the term “two-phase coolant” refers to a composition comprising a solid and a liquid, such as, for example, ice in an aqueous solution.

As used herein, the terms “particulate slurry” and “slurry” refer to a uniform suspension of particles in a solution. A “frozen particulate slurry” refers to a particulate slurry in which the solution is frozen, resulting in the particles suspended in the frozen solution.

As used herein, the term “uniform” is used to describe homogeneity. For example, a uniform particulate slurry comprises a plurality of particles evenly distributed throughout a solution. In contrast, a non-uniform particulate slurry may comprise particles that have risen in the solution due to buoyancy, such as, for example, when ice floats on the surface of liquid water. Non-uniform particulate slurries may also form when particles agglomerate due to the constant phase change at the solid-liquid boundary.

In one aspect, the present invention relates to a frozen particulate slurry. In at least one embodiment, the frozen particulate slurry may comprise a plurality of particles suspended in a frozen solution.

According to at least one embodiment, the plurality of particles comprise a first component. The particles of the first component may be substantially pure. For example, the particles may comprise at least 90% of the first component, such as, at least 95%, at least 97%, at least 98%, at least 99%, at least 99.5%, at least 99.9%, or greater purity. In at least one embodiment, the particles consist essentially of the first component. In at least one further embodiment, the particles consist of the first component.

In at least one embodiment, the frozen solution comprises a mixture of the first component and a second component. The first component and the second component may be capable of forming a eutectic mixture, i.e., a mixture of the first and second component that has a lower freezing point than any other mixture of those same ingredients. For example, sodium chloride and water form a eutectic mixture when the sodium chloride is present in the water in an concentration of about 23.3 wt %, at which concentration, the eutectic mixture has a freezing point of about −21.2° C.

As used herein, the terms “first component” and “second component” are not intended to limit the invention to two compounds. A component may comprise more than one compound. In general, the term “first component” is used to denote the compound or compounds that have the highest freezing point and will freeze first as the temperature of a composition comprising the first and second components is cooled. The term “second component” is used to describe the compound or compounds that depress the freezing point of a mixture of the first and second components, such as sodium chloride depresses the freezing point of an aqueous sodium chloride solution. Other components, such as a “third component,” a “fourth component,” etc., may also be present.

In at least one embodiment, the first component comprises, consists essentially of, or consists of water.

In at least one embodiment, the second component comprises, consists essentially of, or consists of salt and/or sugar. The salt and sugar may be selected from any pharmaceutically acceptable salt or sugar.

Non-limiting examples of salts that may be used in accordance with the present invention include sodium chloride, sodium phosphate, sodium bicarbonate, potassium chloride, potassium phosphate, magnesium chloride, calcium chloride, calcium phosphate, and sulfates. Other salts, including biocompatible salts, known in the art may also be used in accordance with the present invention.

Examples of sugars that may be used include, but are not limited to, dextrose, glucose, sucrose, hydroxyethyl starch (aka, hetastarch), dextran, mannitol, lactate, and pyruvate. Sugars may also include other glucose metabolites commonly administered.

The composition may also comprise other compounds known in the art which are capable of forming eutectic mixtures.

In at least one embodiment, the particulate slurry is formed from a saline solution comprising sodium chloride in water. The sodium chloride may be present in an amount sufficient to provide commonly used saline solutions, such as, for example 0.45 w/v %, 0.90 w/v %, 3.0 w/v %, 5.0 w/v %, or 7.5 w/v % sodium chloride. Other concentrations of sodium chloride may also be used.

In at least one embodiment, a particulate slurry may be formed using any method known in the art, such as, for example, the methods disclosed in U.S. Pat. No. 8,117,854. A saline solution, such as, for example, a saline solution comprising 0.90 w/v % sodium chloride, may be cooled to a temperature below 0° C., the freezing point of water. As the water begins to freeze, the solution may be agitated, such as by stirring, to promote the formation of small ice particles. The particles that form are substantially pure ice. As the ice continues to freeze, the concentration of sodium chloride in the remaining solution increases. Once the concentration of sodium chloride in the solution reaches the eutectic point, i.e., about 23.3 wt/%, the sodium chloride and water form a eutectic mixture and no further ice freezes out of solution. Continued cooling below the eutectic temperature, i.e., cooling below about −21.2° C., will allow the eutectic mixture to freeze and form a solid. The resulting frozen particulate slurry comprising substantially pure ice particles suspended in the eutectic solid formed by the sodium chloride/water solution.

In at least one embodiment, the particles have an average diameter of 1 mm or less, such as, for example, 750 μm or less, 500 μm or less, 350 μm or less, 250 μm or less, 200 μm or less, 150 μm or less, 100 μm or less, or smaller. In at least one embodiment, at least 50% of the particles have an average diameter less than the stated size, such as, for example, at least 75% of the particles are less than the stated size, or at least 90% of the particles are less than the stated size.

The frozen particulate slurry may have a long shelf-life when stored at a temperature below the freezing point of the frozen particulate slurry, e.g., below the eutectic temperature. For example, a frozen particulate slurry comprising sodium chloride and water may be stored for time periods longer than 1 month, longer than 3 months, longer than 6 months, longer than 1 year, or even longer.

One aspect of the present invention relates to a method of freezing a composition.

In at least one embodiment, a composition comprising a first component and a second component are provided. The first component and second component are capable of forming a eutectic mixture. In at least one embodiment, the first component is present in excess over the amount required to form a eutectic mixture with the second component. The composition may be cooled to a temperature below the freezing point of the first component but above the eutectic temperature of the eutectic mixture. The composition is cooled for at least a time sufficient to allow a portion of the first component, i.e., at least a portion of the first component in excess over the eutectic content, to freeze.

In at least one embodiment, the composition is agitated, such as by stirring, as the first component freezes to promote the formation of particles.

According to at least one embodiment, the composition is cooled at a temperature below the freezing point of the first component and above the eutectic temperature for a time sufficient to freeze the first component and form a eutectic mixture from the remaining solution.

In at least one embodiment, once at least a portion of the first component has frozen and formed particles, the composition may be cooled to a temperature below the eutectic temperature to allow the remaining solution to freeze.

In accordance with at least one embodiment, the method further comprises scraping ice off the walls of the container to prevent ice-buildup (i.e., ice fouling) on the walls.

According to at least one embodiment, the composition is not cooled below the eutectic temperature until the remaining solution has formed a eutectic mixture.

The composition may be agitated or stirred, intermittently or continuously, during the freezing of the first component to promote the formation of particles and prevent the formation of large pieces of frozen first component and/or to reduce the agglomeration of already formed particles.

In at least one embodiment, the composition is agitated or stirred until the composition is sufficiently cool to solidify and form a uniform suspension of particles in the frozen eutectic solid.

Another aspect of the present invention relates to thawing a frozen particulate slurry.

In at least one embodiment, a frozen particulate slurry, such as a frozen particulate slurry formed in accordance with the present disclosure is thawed to melt at least a portion of the eutectic solid and form a particulate slurry.

According to at least one embodiment, the frozen particulate slurry is heated to melt at least a portion of the eutectic solid. Heating the frozen particulate slurry may comprise evenly providing heat throughout the entire frozen particulate slurry. For example, a microwave, or any other heat source capable of heating the entire frozen particulate slurry at once, may be used. In at least one embodiment, low power microwaves are intermittently radiated, or pulsed, on the frozen particulate slurry to promote even heating of the eutectic solid portions of the frozen particulate slurry.

Once the eutectic solid portions of the frozen particulate slurry have melted sufficiently, the particulate slurry may be stirred. Stirring may prevent separation of the phases and/or agglomeration of the particles in the slurry.

In at least one embodiment, the particulate slurry, once it is fluid enough, may be subjected to grinding to reduce the particle size.

In at least one embodiment, additional fluid may be added to the frozen particulate slurry before it is thawed, or additional fluid may be added to the frozen particulate slurry as it is being thawed or after it has been thawed. In at least one embodiment, additional fluid is added to the particulate slurry during or after grinding the particulate slurry in order to chemically smooth the ice particles. Without wishing to be limited by theory, it is believed that adding fluid to the particulate slurry and/or stirring may assist in rounding the particles.

In at least one embodiment, more spherical particles may be preferred. In at least one embodiment, additional fluid is added to the particulate slurry and/or the particulate slurry is stirred until particles have a circularity of at least 50%, such as, for example, at least 75%, at least 90%, at least 95%, or greater.

In at least one embodiment, the torque of the stirrer and/or grinder is measured by a torque sensor to determine the consistency and/or particle size of the particulate slurry. Once the torque sensor determines the consistency and/or particle size of the particulate slurry is within a pre-determined range, the stirrer and/or grinder may be stopped.

In at least one embodiment, the frozen particulate slurry comprises a mixture of water with salt and/or sugar. The frozen particulate slurry may have a salt and/or sugar concentration less than the desired salt and/or sugar concentration of the particulate slurry. The method for thawing the frozen particulate slurry may comprise a step of adding additional fluid to the particulate slurry to modify the solute and ice concentration of the slurry. A frozen particulate slurry comprising a low total saline concentration may be thawed and a concentrated fluid saline solution may be added to bring the concentration up to the desired level. For example, a frozen particulate slurry comprising an overall 0.45 w/v % sodium chloride content, may be thawed and a liquid saline solution having a higher sodium chloride content, such as, for example, 3.0 w/v %, 4/5 w/v %, 7.0 w/v %, or 9.0 w/v %, may be added to the particulate slurry to bring the concentration up to 0.90 w/v %.

The additional fluid may comprise the same components as the particulate slurry, or the additional fluid may comprise components different from the components of the particulate slurry. In at least one embodiment, the additional fluid comprises the same components as the particulate slurry.

In at least one embodiment, the additional fluid may comprise at least one compound to supplement or augment the particulate slurry. In at least one embodiment, the additional fluid may be selected based on the purpose for which the particulate slurry is being used.

The additional fluid may comprise any commonly delivered compound. For example, biocompatible salts, carbohydrates, lipids, proteins, metabolic substrates, and surfactants (e.g., exogenous surfactants) may be used in accordance with the present invention. Non-limiting examples of compounds that may be used in accordance with the present invention include, for example, sodium chloride, sodium phosphate, potassium chloride, potassium phosphate, sodium bicarbonate, dextrose, glucose, sucrose, and other commonly administered glucose metabolites, hetastarch, dextran, polyethylene, polyethylene oxide, polyethylene-polyethylene oxide block copolymers, lactate, pyruvate, mannitol, ethyl-pyruvate, magnesium chloride, calcium chloride, calcium phosphate, sulfates, biological phospholipids, cardiolipin, phosphatidic acid, posphatidylcholine, phosphatidylserine, phosphatidylglycerol, amino acids, glysine, nucleotides, NADH, NADPH, FADH2, FMNH, nicotinamide, adenosine, as well as any combination thereof.

A further aspect of the present invention relates to an apparatus for the controlled thawing of a frozen particulate slurry to form a particulate slurry.

In at least one embodiment, the apparatus comprises a container for holding the particulate slurry, a heat source, and an agitator for mixing the particulate slurry, wherein the heat source is capable of evenly providing heat throughout the particulate slurry.

In at least one embodiment, the heat source comprises a microwave. The microwave may be capable of intermittently radiating the particulate slurry.

In at least one embodiment, the container is insulated. According to at least one embodiment, the apparatus is insulated. In at least one embodiment, both the container and apparatus are insulated.

According to at least one embodiment, the agitator is a stirrer. The stirrer may be configured to turn on once the temperature of the particulate slurry rises above the eutectic temperature of the eutectic mixture in the particulate slurry, or to turn on once the particulate slurry has thawed sufficiently to be stirred.

In at least one embodiment, the agitator comprises a scraper. The scraper may be adapted to scrape the walls of the container to prevent fouling caused by solid buildup on the walls of the container. In at least one further embodiment, the agitator comprises a stirrer and a scraper.

In at least one embodiment, the apparatus comprises a torque sensor that measures the torque of the stirrer. The torque sensor may provide a signal once the torque reaches a pre-determined level, such as when the particulate slurry has reached the desired consistency.

In accordance with at least one embodiment, the apparatus comprises a grinder capable of rounding the particles. The grinder may be selected from any grinder known in the art capable of rounding the particles in the particulate slurry, such as, for example, a burr grinder.

The apparatus may also comprise a fluid inlet to allow additional fluid to be added to the particulate slurry. The fluid inlet may allow for external addition of fluid, or the additional fluid may be contained within the apparatus for adding to the particulate slurry.

According to at least one embodiment, the apparatus may comprise a chiller to maintain the particulate slurry at a pre-determined temperature.

In at least one embodiment, the apparatus comprises an outlet for delivering the particulate slurry. The apparatus may further comprise a pump coupled to the outlet for controlling the delivery of the particulate slurry.

In accordance with at least one embodiment, the apparatus may also comprise a delivery device adapted for dispensing the particulate slurry. For example, the delivery device may comprise a tube or hose, which may be insulated. The delivery device may comprise a valve for controlling the flow of the particulate slurry. Other appropriate fittings and connectors are known in the art.

The apparatus in accordance with the present invention may be modular, such that it can be adapted to fit new or existing equipment. For example, the apparatus may be incorporated into equipment for performing robotic, laparoscopic, endoscopic, and catheter-based procedures. The apparatus may be adapted to provide the particulate slurry through known delivery methods, such as, for example, orally, nasally, rectally, or topically. The apparatus may be a portable device or built in to an existing structure. In at least one embodiment, the apparatus is a portable device, such as, for example, a device adapted for use on an ambulance.

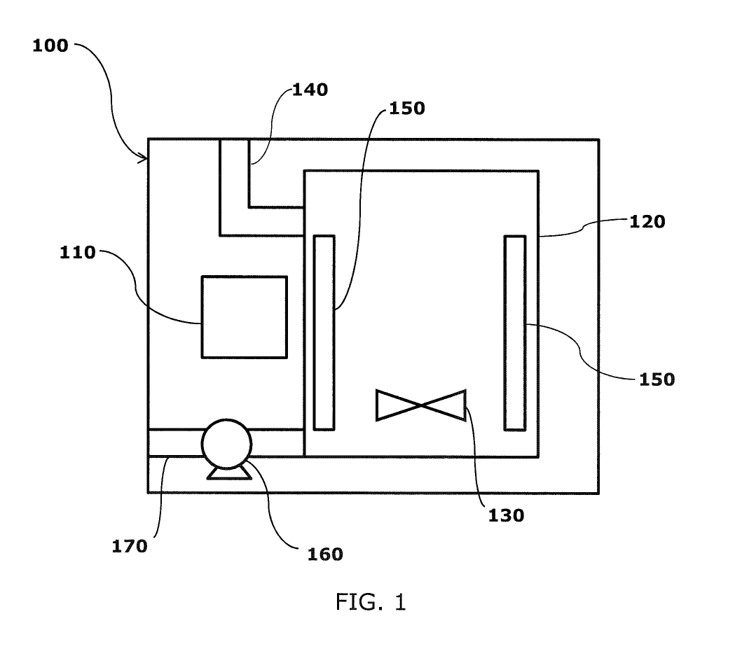

One example of an apparatus according to an embodiment of the present invention is shown in FIG. 1. In FIG. 1, apparatus 100 comprises a heat source 110 and a container 120. A stirrer 130 is present in container 120. Container 120 also comprises grinder 150. Although shown on the side of container 120, grinder 150 may be located in any position conducive to rounding the particles, such as the bottom of container 120 or within the interior of container 120. A fluid inlet 140 provides a channel for additional fluid to be added to container 120. An outlet 170 and pump 160 are connected to the container 120 for delivering the particulate slurry. Outlet 170 may be adapted to connect to a suitable delivery device.

An aspect of the present invention also relates a method of inducing therapeutic hypothermia in a patient and the use of the particulate slurry of the present disclosure for inducing therapeutic hypothermia in a patient.

The use of therapeutic hypothermia may be indicated, for example, in cardiac arrest, stroke, neuro protection (e.g., open or closed brain surgery), spine and spine trauma, cardiac surgery (e.g., open chest or closed chest, including robotic and laparoscopic closed chest surgery). catheter-based coronary intervention (e.g., angioplasty, stenting, atherectomy, electrophysiology, etc.), gastric lavage, orthopedic and orthopedic trauma, cardiac electrophysiology (e.g., open chest, closed chest, and catheter based, including RF ablation/MAZE), partial nephrectomy (e.g., endoscopic, laparoscopic, and robotic, as well as open surgery), no blood flow surgeries, poisoning, diabetic coma, burns, traumatic brain injury, trauma, organ transplant (e.g., harvest, shipment, and implant),and other situations where ischemia is followed by reperfusion. Therapeutic hypothermia may also be used in any other organ surgery to reduce bleeding and/or cross clamp time.

In at least one embodiment, therapeutic hypothermia is induced in a patient by heating a frozen particulate slurry comprising particles of a first component surrounded by a eutectic solid comprising the first component and a second component, wherein the heating melts at least a portion of the eutectic solid and forms a particulate slurry comprising the particles of the first component in a solution of the first component and the second component, and then administering the particulate slurry to the patient in an amount sufficient to locally or systemically induce therapeutic hypothermia.

In at least one embodiment, therapeutic hypothermia is induced in a patient undergoing a nephrectomy, such as, for example, an endoscopic partial nephrectomy, by locally applying a particulate slurry to the kidney of the patient. In at least one embodiment, the particulate slurry is an ice particulate slurry comprising water and sodium chloride.

In at least one embodiment, a particulate slurry is delivered to a patient's stomach to induce therapeutic hypothermia. Then, cardiopulmonary resuscitation (CPR) is performed. In at least one embodiment, at least 1 L of particulate slurry is delivered to the patient's stomach. In at least one further embodiment, at lest 2 L of particulate slurry is delivered to the patient's stomach. According to at least one embodiment, the particulate slurry is an ice particulate slurry comprising water and sugar, such as, for example, dextrose.

The present invention is not limited to the embodiments described and exemplified above, but is capable of variation and modification within the scope and range of equivalents of the appended claims.

I claim:

1. A composition comprising a plurality of particles suspended in a eutectic solid;
wherein the plurality of particles consisting essentially of a first component;
wherein the eutectic solid comprises a eutectic mixture of the first component and a second component;
wherein the plurality of particles are uniformly distributed in the eutectic solid; and
wherein the composition is at a temperature below the eutectic temperature of the eutectic mixture.

2. The composition of claim 1, wherein the first component is water and the second component is chosen from a salt and a carbohydrate.

3. The composition of claim 1, wherein the first component is water and the second component is sodium chloride, wherein the sodium chloride is present in the composition in an amount ranging from about 0.10 w/v % to about 7.5 w/v %.

4. The composition of claim 3, further comprising at least one compound chosen from salts, carbohydrates, lipids, proteins, surfactants, metabolic substrates, amino acids, and nucleotides.

5. A method of freezing a composition comprising:
cooling a composition comprising a first component and a second component, wherein the first component and second component are capable of forming a eutectic mixture and the first component has a freezing point higher than the eutectic temperature of the eutectic mixture, to a temperature below the freezing point of the first component and above the eutectic temperature for a time sufficient to freeze at least a portion of the first component and form a particulate slurry comprising particles consisting essentially of the first component; and
cooling the particulate slurry to a temperature below the eutectic temperature to freeze the remainder of the composition.

6. The method of claim 5, further comprising agitating the composition during the step of cooling the composition to a temperature below the freezing point of the first component and above the eutectic temperature to reduce agglomeration of frozen particles of the first component.

7. The method of claim 6, wherein agitating the composition comprises stirring the composition continuously during the step of cooling the composition to a temperature below the freezing point of the first component and above the eutectic temperature to reduce agglomeration of frozen particles of the first component.

8. The method of claim 6, wherein the composition is in a container having at least one wall, and the method further comprises scraping the at least one wall of the container to prevent ice fouling.

9. The method of claim 5, wherein the composition is cooled to a temperature below the freezing point of the first component and above the eutectic temperature for a time sufficient to form a eutectic mixture comprising the first component that has not frozen and the second component.

10. The method of claim 5, wherein the step of cooling the particulate slurry to a temperature below the eutectic temperature to freeze the remainder of the composition comprises flash freezing the remainder of the composition.

11. A method of thawing a frozen particulate slurry comprising:
heating a frozen particulate slurry comprising particles of a first component surrounded by a eutectic solid comprising the first component and a second component, wherein the heating melts at least a portion of the eutectic solid and forms a particulate slurry comprising the particles of the first component in a solution of the first component and the second component.

12. The method of claim 11, wherein heating the frozen particulate slurry comprises providing heat evenly throughout the frozen particulate slurry with discontinuous pulses of microwaves.

13. The method of claim 11, further comprising stirring the particulate slurry with a stirrer and measuring the torque of the stirrer during the stirring of the particulate slurry.

14. The method of claim 13, wherein the particulate slurry is stirred until the torque of the stirrer reaches a pre-determined value indicative of the consistency of the particulate slurry.

15. The method of claim 11, further comprising reducing the size of the particles of the first component by grinding the particulate slurry and/or adding a fluid to the particulate slurry.

16. The method of claim 11, further comprising adding an additional fluid to the particulate slurry, wherein the additional fluid comprises at least one compound chosen from salts, carbohydrates, lipids, proteins, surfactants, metabolic substrates, amino acids, and nucleotides.

17. An apparatus for controlled thawing of a particulate slurry comprising:
a container for holding the particulate slurry;
a heat source; and
an agitator for mixing the particulate slurry;
wherein the heat source is capable of providing heat evenly throughout the particulate slurry.

18. The apparatus of claim 17, further comprising a grinder for reducing the size of the particles in the particulate slurry.

19. The apparatus of claim 17, wherein the heat source is a microwave.

20. The apparatus of claim 17, wherein the agitator comprises a stirrer, a scraper, and a torque sensor to measure the torque of the stirrer.

21. A method of inducing therapeutic hypothermia in a patient, comprising:
heating a frozen particulate slurry comprising particles of a first component surrounded by a eutectic solid comprising the first component and a second component, wherein the heating melts at least a portion of the eutectic solid and forms a particulate slurry comprising the particles of the first component in a solution of the first component and the second component; and
administering the particulate slurry to the patient in an amount sufficient to locally or systemically induce therapeutic hypothermia.
